(19) United States
(12) Patent Application Publication
(10) Pub. No.: US 2023/0240610 A1
(43) Pub. Date: Aug. 3, 2023

(54) IN-EAR MOTION SENSORS FOR AR/VR APPLICATIONS AND DEVICES

(71) Applicant: Meta Platforms Technologies, LLC, Menlo Park, CA (US)

(72) Inventors: Morteza Khaleghimeybodi, Bothell, WA (US); Barry David Silverstein, Kirkland, WA (US); Nils Thomas Fritiof Lunner, Redmond, WA (US); John Rumsfeld, San Francisco, CA (US); Andrew John Ouderkirk, Kirkland, WA (US); Joshua Weiner, Great Neck, NY (US)

(21) Appl. No.: 18/069,065

(22) Filed: Dec. 20, 2022

Related U.S. Application Data
(60) Provisional application No. 63/305,932, filed on Feb. 2, 2022.
(60) Provisional application No. 63/356,851, filed on Jun. 29, 2022.
(60) Provisional application No. 63/356,860, filed on Jun. 29, 2022.
(60) Provisional application No. 63/356,864, filed on Jun. 29, 2022.
(60) Provisional application No. 63/356,872, filed on Jun. 29, 2022.
(60) Provisional application No. 63/356,877, filed on Jun. 29, 2022.
(60) Provisional application No. 63/356,883, filed on Jun. 29, 2022.

(51) Int. Cl. A61B 5/00; A61B 5/0205

(52) U.S. Cl. CPC A61B 5/6817; A61B 5/0205; A61B 2562/0219

(57) ABSTRACT

An in-ear device for immersive reality applications is provided. The device includes an in-ear fixture configured to fit in an ear canal of a user, a motion sensor mounted on the in-ear fixture and configured to provide a motion signal indicative of an inner body motion or a bulk body motion of the user, a speaker coupled to provide an audio signal to the user, and a processor that is coupled to an augmented reality headset, the processor configured to identify a health condition of the user based on the motion signal. A method for using the above device, a memory and a processor for storing and executing instructions to perform the method are also provided.

### CROSS-REFERENCE TO RELATED APPLICATIONS

The present disclosure is related and claims priority under 35 U.S.C. § 119(e) to U.S. Prov. Appln. No. 63/305,932, entitled IN-EAR BIO-SENSING FOR AR/VR APPLICATIONS AND DEVICES, filed on Feb. 2, 2022, to U.S. Prov. Appln. No. 63/356,851, entitled IN-EAR ELECTRODES FOR AR/VR APPLICATIONS AND DEVICES, to U.S. Prov. Appln. No. 63/356,860, entitled IN-EAR OPTICAL SENSORS FOR AR/VR APPLICATIONS AND DEVICES, to U.S. Prov. Appln. No. 63/356,864, entitled IN-EAR MOTION SENSORS FOR AR/VR APPLICATIONS AND DEVICES, to U.S. Prov. Appln. No. 63/356,872, entitled IN-EAR TEMPERATURE SENSORS FOR AR/VR APPLICATIONS AND DEVICES, to U.S. Prov. Appln. No. 63/356,877, entitled IN-EAR MICROPHONES FOR AR/VR APPLICATIONS AND DEVICES, to U.S. Prov. Appln. No. 63/356,883, entitled IN-EAR SENSORS AND METHODS OF USE THEREOF FOR AR/VR APPLICATIONS AND DEVICES, all filed on Jun. 29, 2022, to Morteza KHALEGHIMEYBODI, et al., the contents of which applications are hereby incorporated by reference in their entirety, for all purposes.

### BACKGROUND

### Field

The present disclosure is related to in-ear motion sensors for use in virtual reality and augmented reality environments and devices. More specifically, the present disclosure is related to motion sensors configured to monitor movement from the inner body of users of in-ear devices for immersive reality applications.

### Related Art

Current in-ear devices (e.g., hearing aids, hearables, headphones, earbuds, and the like) for mobile and immersive applications are typically bulky and uncomfortable for the user. Adding health sensing capabilities to in-ear devices is hindered by the small form factors desirable in such devices and the complex data processing and analysis involved.

### SUMMARY

In a first embodiment, a device includes an in-ear fixture configured to fit in an ear canal of a user, a motion sensor mounted on the in-ear fixture and configured to provide a motion signal indicative of an inner body motion or a bulk body motion of the user, a speaker coupled to provide an audio signal to the user, and a processor that is coupled to an augmented reality headset, the processor configured to identify a health condition of the user based on the motion signal.

In a second embodiment, a computer-implemented method includes receiving, from a motion sensor in an in-ear device, a motion signal indicative of an inner body motion or a bulk body motion of a user of the in-ear device, forming a waveform with the motion signal, and identifying a health condition of the user based on the waveform.

In a third embodiment, a non-transitory, computer-readable medium stores instructions which, when executed by a processor, cause a computer to perform a method. The method includes receiving, from a motion sensor in an in-ear device, a motion signal indicative of an inner body motion or a bulk body motion of a user of the in-ear device, forming a waveform with the motion signal, and identifying a health condition of the user based on the waveform.

In yet other embodiments, a system includes a first means to store instructions and a second means to execute the instructions and cause the system to perform a method. The method includes receiving, from a motion sensor in an in-ear device, a motion signal indicative of an inner body motion or a bulk body motion of a user of the in-ear device, forming a waveform with the motion signal, and identifying a health condition of the user based on the waveform.

These and other embodiments will become clear to one of ordinary skill in the art in view of the following.

### BRIEF DESCRIPTION OF THE FIGURES

FIG. 1 illustrates an AR headset and an in-ear monitor (IEM) in an architecture configured to assess a user's health, according to some embodiments.

FIG. 2 illustrates an augmented reality ecosystem including wearable devices in the ear and wrist to assess a user's health, according to some embodiments.

FIGS. 3A-3C illustrate different embodiments of an in-ear monitor (IEM), according to some embodiments.

FIG. 4 illustrates a motion waveform overlapped with an electrocardiogram (ECG) signal provided by an in-ear electrode, or any electrode disposed on a wearable device, according to some embodiments.

FIG. 5 illustrates a motion waveform obtained with a motion sensor in an IEM to determine a heart rate of a user, according to some embodiments.

FIG. 6 is a flow chart illustrating steps in a method for using motion sensors in an in-ear monitor for assessing the health of a user of a headset or smart glass, according to some embodiments.

FIG. 7 is a block diagram illustrating an exemplary computer system with which headsets and other client devices, and the method in FIG. 6 be implemented, according to some embodiments.

In the figures, elements having the same or similar reference numeral are associated with the same or similar features and attributes, unless explicitly stated otherwise.

### DETAILED DESCRIPTION

In the following detailed description, numerous specific details are set forth to provide a full understanding of the present disclosure. It will be apparent, however, to one ordinarily skilled in the art, that the embodiments of the present disclosure may be practiced without some of these specific details. In other instances, well-known structures and techniques have not been shown in detail so as not to obscure the disclosure.

### General Overview

Head-worn devices (e.g., devices worn on head including but not limited to hearables, smart glasses, AR/VR headsets and smart glasses, etc.) offer opportunities to access valuable health information.

The ear (e.g., the ear canal and ear concha and pinna) has close proximity to the brain, to body chemistry, and blood vessels indicative of brain activity and cardio-respiratory activity, and inner body temperature. More specifically, sensors can be placed inside the ear canal or around the ear (in the case of AR/VR headsets or smart glasses) to sense the brain, heart, and ocular electrophysiological activities (e.g., electro-encephalography, EEG, electro-cardiography, ECG, electro-oculography, EOG, electrodermal activity, EDA, and the like); or to sense vital signs (heart rate, breathing rates, blood pressure, body temperature, and the like); or to sense the body chemistry (e.g., blood alcohol level, blood glucose estimation, and the like).

Motion sensors, optical sensors, microphones, thermometers, and electrodes in embodiments as disclosed herein may be used in EOG, ECG, or EEG measurements, e.g., for determining auditory attention; heart rate estimation, breathing rate, and the like, Auditory Steady State Response-ASSR-, or auditory brainstem response-ABR-. In some embodiments, in-ear electrodes as disclosed herein may be useful to measure resting state electric oscillations (alpha waves in an EEG) that can track relaxation/activity. With the combination of other measurements (e.g., photoplethysmography, PPG and/or electrocardiography, ECG), a new branch of diagnostic possibilities is open. In-ear EEG measurements can be applied to track user attention (e.g., distinguishing between attention focus from eye gaze direction).

Methods and devices disclosed herein include motion, optical, and acoustical sensors, chemical sensors, and temperature sensors, in and around the ears of AR/VR headset users, in combination with software correlation of the signals provided by the above sensors to generate comprehensive diagnostics and health evaluation of the user.

Some of the features disclosed herein include in-ear or head-worn body temperature sensing using infrared sensing and spectroscopy techniques. In some embodiments, the contact area for sensors as disclosed herein include the in-ear canal (like an in-ear earbud) and within the conchal bowl (in human pinna), areas on top of the human ear (where the glasses sit), and areas in the nose-pad of a headset or smart glass (where glasses sit on the nose). Some measurements may include in-ear or around the ear sensing of glucose level, alcohol sensing, body temperature, blood pressure, and the like. Some embodiments include optical-based pulse transit time (PTT) methodology to estimate blood pressure for a glass/headset device using a combination of optical and electrical signals (e.g., PPG+ECG sensors respectively). Some embodiments obtain user's blood pressure using an optical sensing technique (PPG) in combination with a deep neural network to train a network based using both PPG information and a corresponding ground-truth blood pressure information. Some embodiments include motion-based pulse transit time (PTT) methodology to estimate blood pressure for a glass/headset device using a combination of motion sensor and electrical signals (e.g., IMU+ECG sensors respectively). In some embodiments, PPG signals collected in IEM devices as disclosed herein may be able to estimate the cognitive load on the user with analysis of oxygenated and deoxygenated blood flow (oxy- and deoxy-hemoglobin) to the brain. Some embodiments include sensing alcohol levels through emissions around the ear. Some embodiments incorporate chemical sensing intake around the contact points of the ear. In some embodiments, IEM devices may perform alcohol monitoring and fat burning during user exercise.

### Example System Architecture

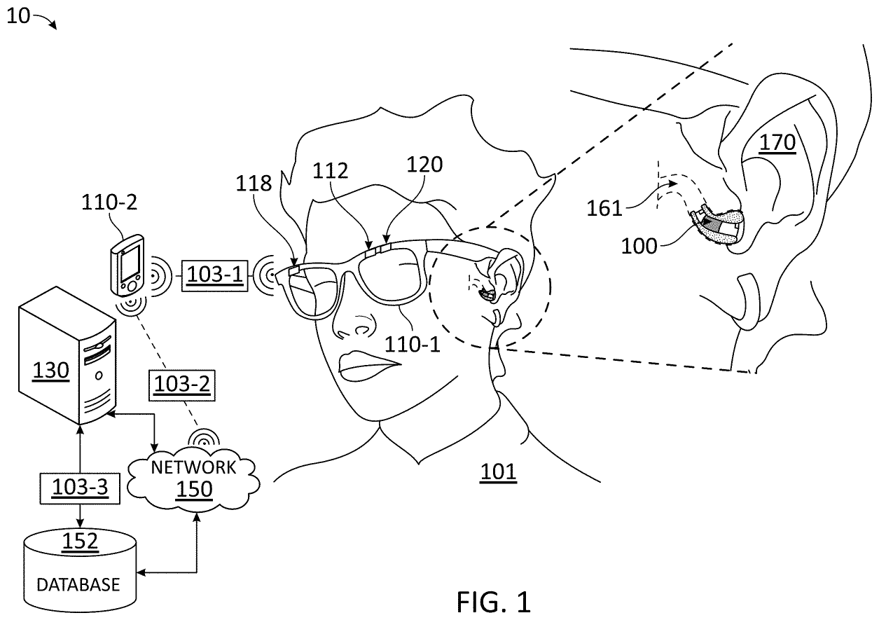

FIG. 1 illustrates an AR headset 110-1 and an in-ear monitor (IEM) 100 in an architecture 10 configured to assess the health of a user 101, according to some embodiments. IEM 100 is inserted in the ear 170 of user 101, reaching the ear canal 161. AR headset 110-1 may include smart glasses having a memory circuit 120 storing instructions and a processor circuit 112 configured to execute the instructions to perform steps as in methods disclosed herein. AR headset 110-1 (or smart glasses) may also include a communications module 118 configured to wirelessly transmit information (e.g., Dataset 103-1) between AR headset 110-1 (and/or in-ear device 100, and/or a smart watch, or combination of the above) and a mobile device 110-2 with the user (AR headset 110-1 and mobile device 110-2 will be collectively referred to, hereinafter, as “client devices 110”). Communications module 118 may be configured to interface with a network 150 to send and receive information, such as dataset 103-1, dataset 103-2, and dataset 103-3, requests, responses, and commands to other devices on network 150. In some embodiments, communications module 118 can include, for example, modems or Ethernet cards. Client devices 110 may in turn be communicatively coupled with a remote server 130 and a database 152, through network 150, and transmit/share information, files, and the like with one another (e.g., dataset 103-2 and dataset 103-3). Datasets 103-1, 103-2, and 103-3 will be collectively referred to, hereinafter, as “datasets 103.” Network 150 may include, for example, any one or more of a local area network (LAN), a wide area network (WAN), the Internet, and the like. Further, the network can include, but is not limited to, any one or more of the following network topologies, including a bus network, a star network, a ring network, a mesh network, a star-bus network, tree or hierarchical network, and the like.

In some embodiments, at least one of the steps in methods as disclosed herein are performed by processor 112, providing dataset 103-1 to mobile device 110-2. Mobile device 110-2 may further process the signals and provide dataset 103-2 to database 152 via network 150. Remote server 130 may collect dataset 103-2 from multiple AR headsets 110-1 and mobile devices 110-2 in the form and perform further calculations. In addition, having aggregated data from a population of individuals, the remote server may perform meaningful statistics. This data cycle may be established provided each of the users involved have consented for the use of de-personalized, or anonymized data. In some embodiments, remote server 130 and database 152 may be hosted by a healthcare network, or a healthcare facility or institution (e.g., hospital, university, government institution, clinic, health insurance network, and the like). Mobile device 110-2, AR headset 110-1, in-ear device 100, and applications therein may be hosted by a different service provider (e.g., a network carrier, an application developer, and the like). Moreover, AR headset 110-1 and mobile devices 110-2 may proceed from different manufacturers. User 101 is ultimately the sole owner of dataset 103-1 and all data derived therefrom (e.g., datasets 103), and so all the data flows (e.g., datasets 103), while provided, handled, or regulated by different entities, are authorized by user 101, and protected by network 150, server 130, database 152, and mobile device 110-2 for privacy and security.

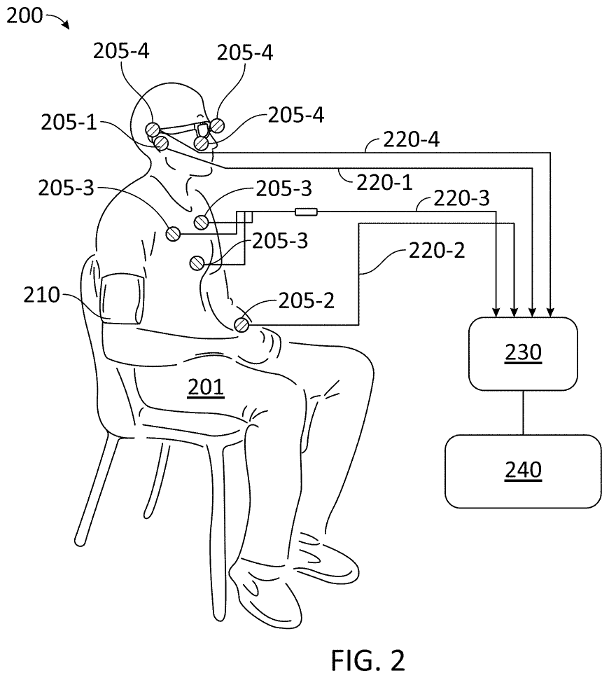

FIG. 2 illustrates an augmented reality ecosystem 200 including wearable devices in the ear 205-1 (e.g., an IEM), wrist 205-2, chest 205-3, and smart glass sensors 205-4 to assess the health of user 201, according to some embodiments. In some embodiments, IEM 205-1 further includes an optical sensor configured to provide an optical signal 220-1 to a processor in a computer 240 via a data acquisition module (DAQ) 230. IEM 205-1 may further include one or more contact electrodes configured to provide an electrical signal to a processor in a computer 240 via a data acquisition module (DAQ) 230. Computer 240 is configured to identify a cardiovascular condition of user 201 based on a first electronic signal from IEM 205-1 and optical signal 220-1. In some embodiments, IEM 205-1 further includes a motion sensor (e.g., an accelerometer, a contact microphone, or an IMU) configured to provide a motion-based signal to computer 240 via DAQ 230. In some embodiments, a pair of IEMs 205 will be placed in both ears and different optical, electrical (electrode), acoustic (microphone), or motion sensors (accelerometer, IMU, contact microphone, etc.) may be placed in either both sides; or in some cases, some sensors may be placed on one side (e.g., the Right side) and some other sensors may be placed on the other side (e.g., Left side). Computer 240 is configured to identify a cardiovascular condition of the user based on a first electronic signal from IEM 205-1 and the motion signal. The optical sensor may be a photo-plethysmography (PPG) sensor and optical signal 220-1 may include a digital or analog signal indicative of a vascular activity inside the ear of user 201. Chest sensors 205-3 and smart glass sensors 205-4 may include ECG sensors to provide a distributed signals 220-3 and 220-4 from one or more areas around the chest and face (e.g., the outside of the ear, the chin, and the nose) of user 201, respectively (or alternatively an ECG can be collected from some electrodes placed on areas on the head or from electrodes placed in IEM 205-1, or electrodes placed on the wrist device 205-2), and a wrist PPG sensor in device 205-2 may provide a separate signal 220-2 for vascular activity around the wrist of user 201. IEM 205-1, wrist sensor 205-2, chest sensors 205-3, and smart glass sensors 205-4 will be collectively referred to, hereinafter, as “wearable devices (and sensors) 205.” Blood pressure (BP) measurements may be obtained with a cuff or cuff-less BP monitor 210 and may also be determined by comparing PPG signals 220-1 and 220-2. Signals 220-1, 220-2, 220-3 and 220-4 (hereinafter, collectively referred to, hereinafter, as “signals 220”) may be collected and digitized by DAQ 230 in computer 240, for processing. In some embodiments, signals 220 and others may be wired, or wireless. In some embodiments, it may be preferable to have wireless signal communication between the different wearable devices 205 with user 201. In some embodiments, wearable devices and sensors 205 may include one or more motion sensors, and the motion-based information collected from the smart glass, the IEM, chest or wrist can be combined to create a more meaningful information.

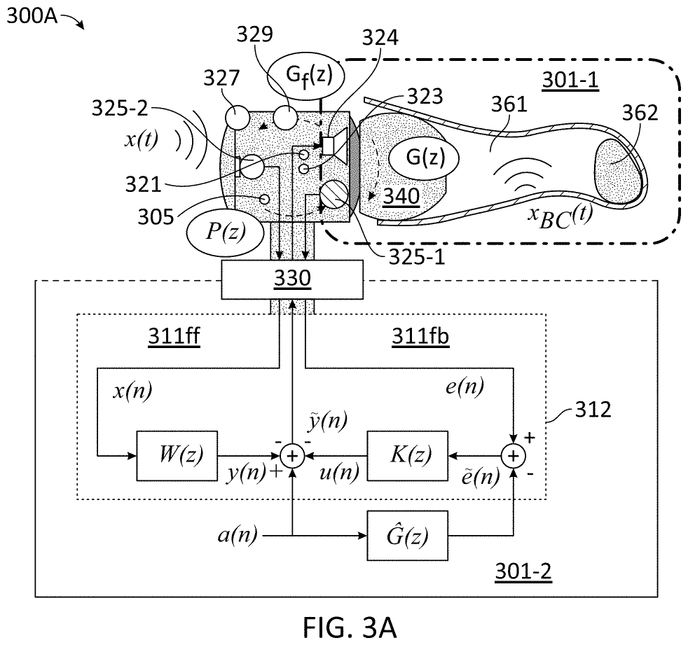

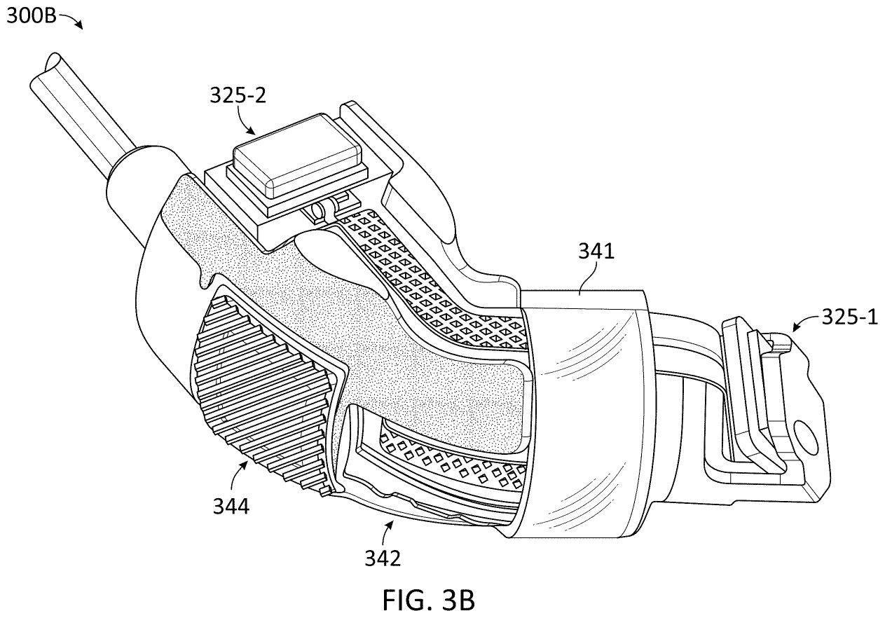

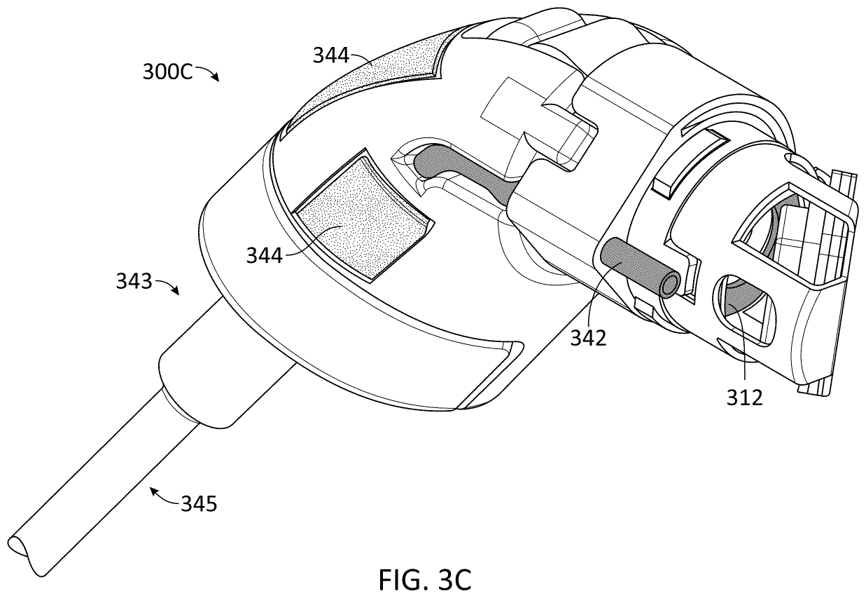

FIGS. 3A-3C illustrate different embodiments of an in-ear monitor (IEM) 300A, 300B, and 300C (hereinafter, collectively referred to as “IEMs 300”), according to some embodiments. IEMs 300 may include a front end 301-1 including sensors and open to ear canal 361 and ear drum 362, and a back end 301-2 including a processor 312. IEMs 300 may include sensors such as: an electrode 305 to sense electrical signals, acoustic sensors 325-1 and 325-2 (e.g., collectively referred hereinafter, as “microphones 325”), motion sensors 327 (e.g., accelerometers, contact microphones, inertial motion units-IMUs, and the like), temperature sensors 329, and optical sensors including an emitter 321 and a detector 323 (e.g., LEDs and PDs in PPG sensors, functional near-infrared fNIR sensors-Fourier transform based, spectroscopic based-). Electrodes 305 may include bio-potential electrodes for applications such as EEG, ECG, EOG, and EDA). In addition, processor 312 may handle at least some of the operations for signal acquisition and control of components and sensors 321, 323, 324 (a speaker), 325-1 (internal microphone), 325-2 (external microphone, hereinafter, collectively referred to as “microphones 325”), 327, and 329 via a digital-to-analog and/or analog-to-digital converter (DAC/ADC) 330. Processor 312 may include a feedforward stage 311ff and a feedback stage 311fb that cooperate to process the signal from the sensors: noise reduction, balancing, filtering, and amplification.

In some embodiments, electrodes 305 include a contact electrode configured to transmit a current from the skin in the ear canal of the user. In some embodiments, an electrode 305 is coated with at least one of a gold layer, a silver layer, a silver chloride layer, or a combination thereof. In some embodiments, electrodes 305 include a capacitive coupling electrode disposed sufficiently close, but not in contact, with the user's skin. In some embodiments, IEMs 300 further include at least a second electrode 305 mounted on in-ear fixture 340, the second electrode 305 configured to receive a second electronic signal from the skin in ear canal 361. In some cases, the in-ear fixture 340 (also known as “eartip”) may be entirely made out of soft conductive materials (e.g., conductive polymers, conductive adhesives, conductive paints, etc.). Accordingly, the entire eartip will be conductive and will act as a soft electrode to collect electrical signals from the skin of the ear-canal. In some embodiments, processor 312 is configured to select the first electronic signal when a quality of the first electronic signal is higher than a pre-selected threshold. In some embodiments, processor 312 is configured to reduce a noise background from the first electronic signal with the second electronic signal. In some embodiments, processor 312 is configured to determine a heart rate of the user from the first electronic signal. In some embodiments, processor 312 is configured to determine a brain activity from the first electronic signal that corresponds to an acoustic stimulus received in the external microphone.

In some embodiments, emitter 321 and detector 323 may be part of a self-mixing interferometer (SMI). An SMI is a compact, low power, inexpensive and sensitive optical device configured to measure reflectivity, including back scatter, as well as displacement of the skin. In some embodiments, a displacement of the skin obtained with an SMI is combined with heart rate measurements (e.g., from PPG sensors, motion sensors or ECG electrodes) to measure blood pressure and heart rate, or even vibration of the eardrum to also act as an internal microphone.

IEMs 300 in the AR headset or smart glasses may include an in-ear fixture 340 configured to hermetically seal an ear canal of a user, a first electrode 305 mounted on in-ear fixture 340 and configured to receive a first electronic signal from a skin in ear canal 361, and an internal microphone 325-1 coupled to receive an internal acoustic signal, propagating through ear canal 361. An acoustic front end includes internal microphone 325-1 configured to detect acoustic waves (x_{BC}(t)) propagated through ear canal 361 and generated by the inner body (e.g., heart rate at about <100 Hz, breathing rate at about 50-1000 Hz, and other sounds in the laryngeal cavity). An external microphone 325-2 is coupled to receive an external acoustic signal x(t), propagating through an environment of the user. In some embodiments, the internal signal x_{BC}(t) in conjunction with the external signal x(t) may be used in acoustic procedures such as audio streaming, hear-through, active noise cancelation (ANC), hearing corrections, virtual presence and spatial audio, call services, and the like. In some embodiments, at least some of the above processes are performed in conjunction between left-ear and right-ear IEM monitors 300. In some embodiments, the internal signal x_{BC}(t) can be captured using the motions sensors placed within the in-ear device. In some other embodiments, a combination of motion sensors 327 and acoustic sensors or microphones 325-1 and 325-2 can be used to collect bi-modal mechanical and acoustic turbulences driven from the heart pulsation.

IEM 300B includes a sealing gasket 341 that separates the inner portion of ear canal 361 from the environment, leaving a back-volume vent including an acoustically resistive mesh 344 for a pressure equalizer (PEQ) tube 342 to vent into resistive mesh 344 (also shown in IEM 300C). The sealed cavity may enable breathing and heart rate monitoring (e.g., isolating the signal from internal acoustic microphone 325-1) at low power usage and with a small form factor.

IEM 300C illustrates processor circuit 312 to identify a cardiovascular condition or a neurologic condition of the user, based on at least one of a first electronic signal, an internal acoustic signal, and an external acoustic signal (e.g., from microphones 325). Some embodiments may include a down cable 345 to electrically couple the IEM with the VR headset or smart glasses, including a strain relief 343.

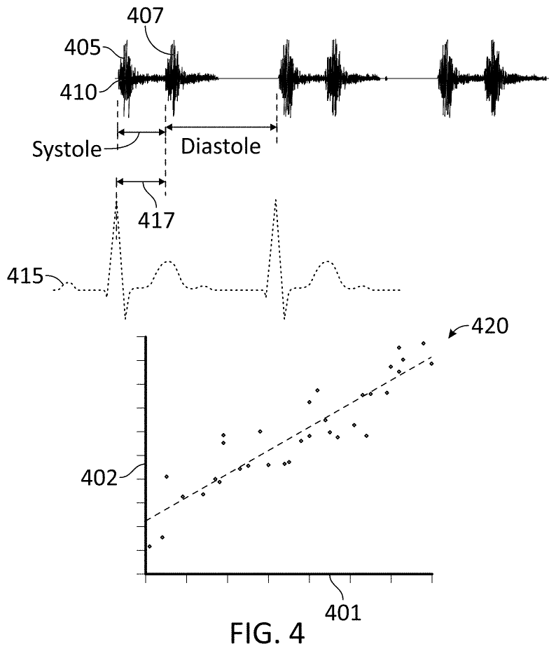

FIG. 4 illustrates a motion waveform 410 overlapped with an ECG signal 415 provided by an in-ear electrode, or any electrode disposed on a wearable device (e.g., a smart watch, or a wristband, and the like), according to some embodiments. ECG signal 415 provides a reference time for the start of a heart pulse, from which a systolic portion 405 and a diastolic portion 407 of motion waveform 410 may be identified. Accordingly, a time lapse 417 between the initial electronic pulse 415 and the diastolic portion 407 may be indicative or have a direct correlation 420 with a blood pressure 401 of the user. Other correlation factors to identify a blood pressure for the user may include a ratio 402 of an amplitude of the systolic portion 405 to the diastolic portion 407.

In some embodiments, the spectral signature of systolic portion 405 and/or diastolic portion 407 may also be indicative of a user's vital signs. It is generally observed that systolic portion 405 includes a narrower frequency bandwidth, while diastolic portion 407 has a broader bandwidth.

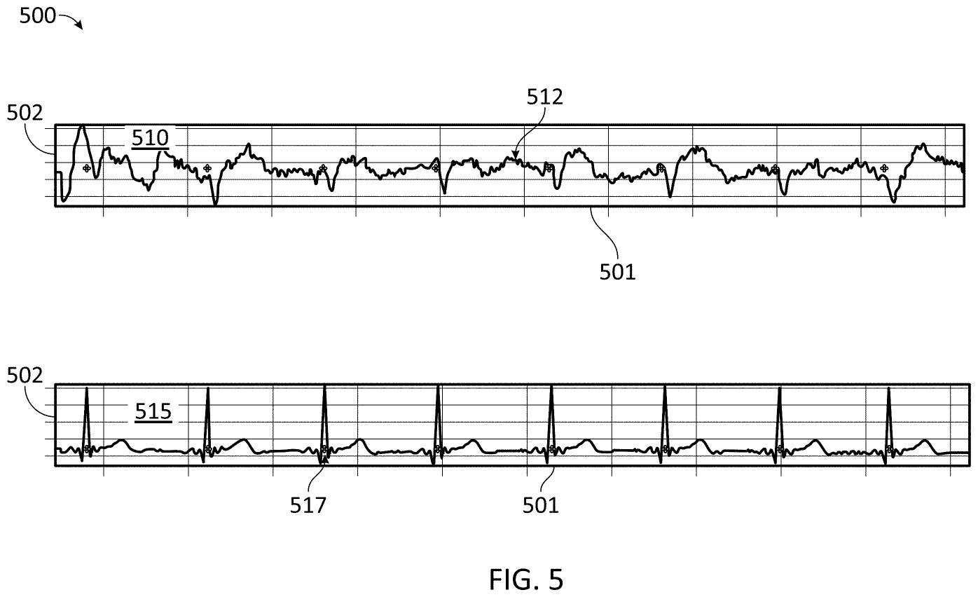

FIG. 5 is a chart 500 illustrating a motion waveform 510 obtained with a motion sensor in an IEM to determine a heart rate of a user, according to some embodiments. Chart 500 includes a time abscissae 501 and a signal value 502. Motion waveform 510 may include the raw signal from the sensor (e.g., when a 1D accelerometer or contact microphone is used); or from a processing pipeline combining motions from multiple axes (e.g., 3D-axes accelerometer or when a 6-DOF or 9-DOF IMU is used). A ground-truth ECG 515 includes R-peaks 517.

Other measurements available from motion waveform 510 may include, in addition to heart rate and breathing rate, and without limitation: step count, pose estimation, and fall detection. Moreover, some embodiments enable blood pressure estimation using the pulse transit time (PTT) technique combining motion waveform 510 and an ECG signal 515.

In some embodiments, motion waveform 510 may retrieve body-borne infrasound and low frequency vibrations associated with a user's vital signs. Signal processing techniques, in conjunction with artificial intelligence (AI) processing, can be used to extract user's vital signs from motion waveform 510 (e.g., heart rate, heart rate variability, breathing rate, and blood pressure).

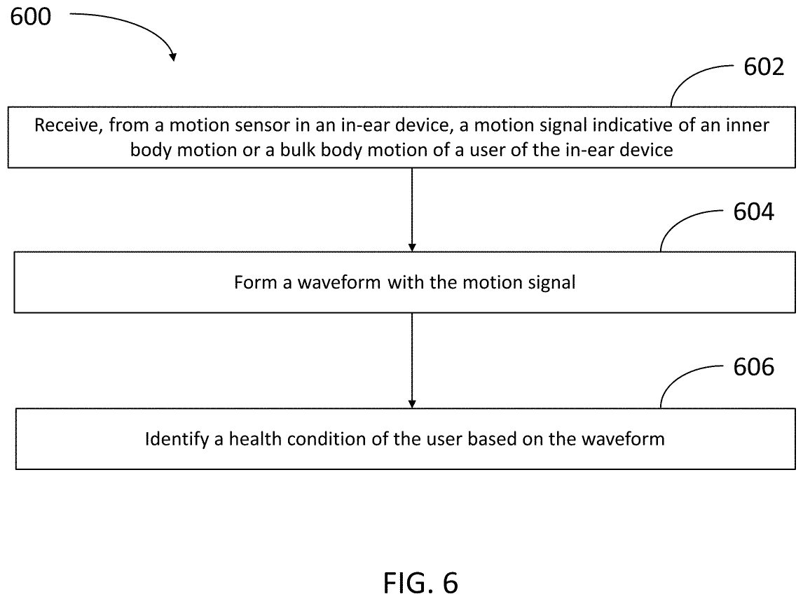

FIG. 6 is a flow chart illustrating steps in a method 600 for using electrodes in an in-ear monitor for assessing the health of a user of a headset or smart glass, according to some embodiments. In some embodiments, at least one or more of the steps in method 600 may be performed by a processor executing instructions stored in a memory in either one of smart glasses or other wearable devices on a user's body part (e.g., ears, head, arm, wrist, leg, ankle, finger, toe, knee, shoulder, chest, back, and the like). In some embodiments, at least one or more of the steps in method 600 may be performed by a processor executing instructions stored in a memory, wherein either the processor or the memory, or both, are part of a mobile device for the user, a remote server, or a database, communicatively coupled with each other via a network (cf., processors 112, 312, and memory 120, client devices 110, server 130, database 152, and network 150). Moreover, the mobile device, the smart glasses, and the wearable devices may be communicatively coupled with each other via a wireless communication system and protocol (e.g., communications module 118, radio, Wi-Fi, Bluetooth, near-field communication-NFC- and the like). In some embodiments, a method consistent with the present disclosure may include one or more steps from method 600 performed in any order, simultaneously, quasi-simultaneously, or overlapping in time.

Step 602 includes receiving, from a motion sensor, a motion signal indicative of an inner body motion or a bulk body motion of a user of the in-ear device. In some embodiments, the motion sensor is part of an in-ear device, or a wearable device attached to a different part of the user's body (e.g., the wrist, shoulder, arm, leg, or ankle, or any combination thereof). In some embodiments, step 602 includes receiving, from a second motion sensor in a second in-ear device, a second motion signal indicative of the inner body motion or the bulk body motion. The second in-ear device may be placed on the opposite side, enabling binaural capturing of information from both left and right ear canals. In some embodiments, step 602 includes receiving, from an electrode, an electronic signal indicative of a cardiovascular activity of the user.

Step 604 includes forming a waveform with the motion signal. In some embodiments, step 604 includes removing at least one of a noise component or an interference from the motion signal with the second motion signal. The motion sensor in the in-ear device is configured to provide a motion-based signal to the processor, wherein the motion waveform may directly come from the raw signal from the sensor (e.g., when a 1D accelerometer or contact microphone is used), or from a processing pipeline combining motions from multiple axes (e.g., 3D-axes accelerometer or when a 6-degree of freedom-DOF- or 9-DOF IMU is used).

Step 606 includes identifying a health condition of the user based on the waveform. In some embodiments, the motion signal is indicative of an inner body motion and step 606 includes determining at least one of a heart rate and a breathing rate of the user. In some embodiments, the motion signal is indicative of a bulk body motion of the user and step 606 includes at least one of detecting a fall of the user, a cough of the user, a sneeze of the user, or determining a step count for the user. In some embodiments, step 606 further includes identifying a systolic heart pulse and a diastolic heart pulse from the waveform, and identifying features in the systolic heart pulse and the diastolic heart pulse. In some embodiments, step 606 includes identifying a systolic heart pulse and a diastolic heart pulse from the waveform, and determining a blood pressure of the user based on an amplitude of the diastolic heart pulse and on an amplitude of the systolic heart pulse. In some embodiments, step 606 includes identifying a systolic heart pulse and a diastolic heart pulse from the waveform, and determining a blood pressure of the user based on a time delay of the diastolic heart pulse relative to the systolic heart pulse. In some embodiments, step 606 includes filtering, with the waveform, one of a noise component and an interference from an audio signal to a speaker mounted in the in-ear device, and providing, with the speaker, the audio signal to the user. In some embodiments, step 606 includes identifying a correlation of the electronic signal with the waveform, and determining one of a heart rate, a breathing rate, or a blood pressure of the user based on the correlation. In some embodiments, step 606 further includes performing a spectral analysis on the waveform to identify a p-wave, a QRS-complex, and a T-wave complex in an electro-cardiogram.

### Hardware Overview

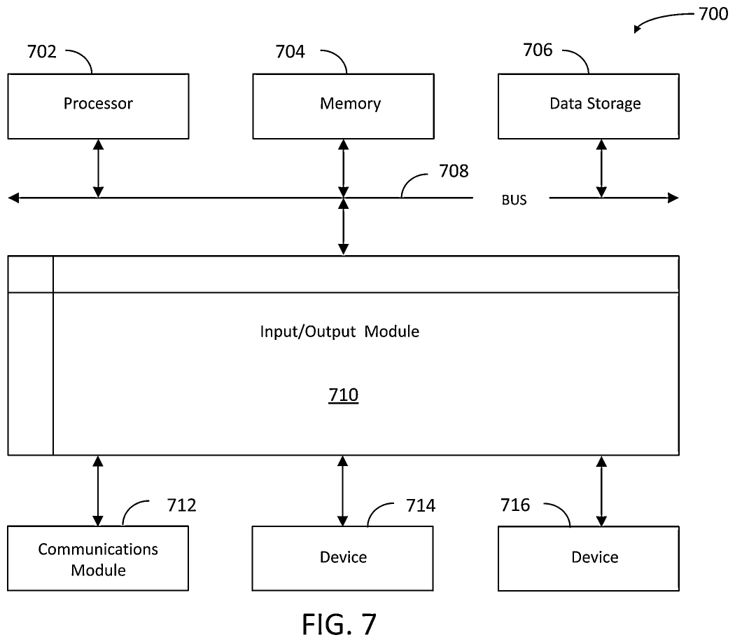

FIG. 7 is a block diagram illustrating an exemplary computer system 700 with which headsets and other client devices 110, and method 600 can be implemented, according to some embodiments. In certain aspects, computer system 700 may be implemented using hardware or a combination of software and hardware, either in a dedicated server, or integrated into another entity, or distributed across multiple entities. Computer system 700 may include a desktop computer, a laptop computer, a tablet, a phablet, a smartphone, a feature phone, a server computer, or otherwise. A server computer may be located remotely in a data center or be stored locally.

Computer system 700 includes a bus 708 or other communication mechanism for communicating information, and a processor 702 (e.g., processors 112) coupled with bus 708 for processing information. By way of example, the computer system 700 may be implemented with one or more processors 702. Processor 702 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 700 can include, in addition to hardware, code that creates an execution environment for the computer program in question, e.g., code that constitutes processor firmware, a protocol stack, a database management system, an operating system, or a combination of one or more of them stored in an included memory 704 (e.g., memory 120), such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled with bus 708 for storing information and instructions to be executed by processor 702. The processor 702 and the memory 704 can be supplemented by, or incorporated in, special purpose logic circuitry.

The instructions may be stored in the memory 704 and implemented in one or more computer program products, e.g., one or more modules of computer program instructions encoded on a computer-readable medium for execution by, or to control the operation of, the computer system 700, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 704 may also be used for storing temporary variable or other intermediate information during execution of instructions to be executed by processor 702.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 700 further includes a data storage device 706 such as a magnetic disk or optical disk, coupled with bus 708 for storing information and instructions. Computer system 700 may be coupled via input/output module 710 to various devices. Input/output module 710 can be any input/output module. Exemplary input/output modules 710 include data ports such as USB ports. The input/output module 710 is configured to connect to a communications module 712. Exemplary communications modules 712 include networking interface cards, such as Ethernet cards and modems. In certain aspects, input/output module 710 is configured to connect to a plurality of devices, such as an input device 714 and/or an output device 716. Exemplary input devices 714 include a keyboard and a pointing device, e.g., a mouse or a trackball, by which a consumer can provide input to the computer system 700. Other kinds of input devices 714 can be used to provide for interaction with a consumer as well, such as a tactile input device, visual input device, audio input device, or brain-computer interface device. For example, feedback provided to the consumer can be any form of sensory feedback, e.g., visual feedback, auditory feedback, or tactile feedback; and input from the consumer can be received in any form, including acoustic, speech, tactile, or brain wave input. Exemplary output devices 716 include display devices, such as an LCD (liquid crystal display) monitor, for displaying information to the consumer.

According to one aspect of the present disclosure, headsets and client devices 110 can be implemented, at least partially, using a computer system 700 in response to processor 702 executing one or more sequences of one or more instructions contained in memory 704. Such instructions may be read into memory 704 from another machine-readable medium, such as data storage device 706. Execution of the sequences of instructions contained in main memory 704 causes processor 702 to perform the process steps described herein. One or more processors in a multi-processing arrangement may also be employed to execute the sequences of instructions contained in memory 704. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., a data server, or that includes a middleware component, e.g., an application server, or that includes a front end component, e.g., a client computer having a graphical consumer interface or a Web browser through which a consumer can interact with an implementation of the subject matter described in this specification, or any combination of one or more such back end, middleware, or front end components. The components of the system can be interconnected by any form or medium of digital data communication, e.g., a communication network. The communication network can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 700 can include clients and servers. A client and server are generally remote from each other and typically interact through a communication network. The relationship of client and server arises by virtue of computer programs running on the respective computers and having a client-server relationship to each other. Computer system 700 can be, for example, and without limitation, a desktop computer, laptop computer, or tablet computer. Computer system 700 can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to processor 702 for execution. Such a medium may take many forms, including, but not limited to, non-volatile media, volatile media, and transmission media. Non-volatile media include, for example, optical or magnetic disks, such as data storage device 706. Volatile media include dynamic memory, such as memory 704. Transmission media include coaxial cables, copper wire, and fiber optics, including the wires forming bus 708. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

As used herein, the phrase “at least one of” preceding a series of items, with the terms “and” or “or” to separate any of the items, modifies the list as a whole, rather than each member of the list (e.g., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The word “exemplary” is used herein to mean “serving as an example, instance, or illustration.” Any embodiment described herein as “exemplary” is not necessarily to be construed as preferred or advantageous over other embodiments. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public, regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

While this specification contains many specifics, these should not be construed as limitations on the scope of what may be described, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially described as such, one or more features from a described combination can in some cases be excised from the combination, and the described combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the described subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately described subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

What is claimed is:

1. A device, comprising:
an in-ear fixture configured to seal an ear canal of a user;
a motion sensor mounted on the in-ear fixture and configured to provide a motion signal indicative of an inner body motion or a bulk body motion of the user;
a speaker coupled to provide an audio signal to the user; and
a processor that is coupled to an augmented reality headset, the processor configured to identify a health condition of the user based on the motion signal.

2. The device of claim 1, wherein the motion sensor is a contact microphone and the motion signal is transmitted through a body of the user into a contact point between the motion sensor and the ear canal of the user.

3. The device of claim 1, wherein the motion sensor is an inertial motion unit and the motion signal is indicative of a bulk body motion of the user including one of a sneeze, a cough, a fall, and a step.

4. The device of claim 1, wherein the motion sensor is a micro-electronic moving system.

5. The device of claim 1, wherein the motion signal is indicative of an inner body motion of the user including one of a heart rate or a respiratory rate.

6. The device of claim 1, further comprising an electrode mounted on the in-ear fixture, the electrode configured to receive a signal indicative of a cardiovascular activity of the user.

7. The device of claim 1, further comprising an electrode mounted on the in-ear fixture, the electrode configured to receive a signal indicative of a neural activity of the user.

8. The device of claim 1, wherein the processor is configured to synchronize a waveform with the motion signal and a waveform with an electronic signal to determine a vital sign of the user.

9. The device of claim 1, wherein the processor is configured to determine a heart rate of the user from the motion signal.

10. The device of claim 1, wherein the processor is configured to remove one of a noise component or an interference from the audio signal to the user based on the motion signal.

11. A computer-implemented method, comprising:
receiving, from a motion sensor in an in-ear device, a motion signal indicative of an inner body motion or a bulk body motion of a user of the in-ear device;
forming a waveform with the motion signal; and
identifying a health condition of the user based on the waveform.

12. The computer-implemented method of claim 11, further comprising receiving, from a second motion sensor in a second in-ear device, a second motion signal indicative of the inner body motion or the bulk body motion, and wherein forming the waveform comprises removing at least one of a noise component or an interference from the motion signal with the second motion signal.

13. The computer-implemented method of claim 11, wherein the motion signal is indicative of an inner body motion and identifying a health condition of the user comprises determining at least one of a heart rate and a breathing rate of the user.

14. The computer-implemented method of claim 11, wherein the motion signal is indicative of a bulk body motion of the user and identifying a health condition of the user comprises at least one of detecting a fall of the user, a cough of the user, a sneeze of the user, or determining a step count for the user.

15. The computer-implemented method of claim 11, further comprising identifying a systolic heart pulse and a diastolic heart pulse from the waveform, wherein identifying a health condition of the user based on the waveform comprises identifying features in the systolic heart pulse and the diastolic heart pulse.

16. The computer-implemented method of claim 11, further comprising identifying a systolic heart pulse and a diastolic heart pulse from the waveform, wherein identifying a health condition of the user based on the waveform comprises determining a blood pressure of the user based on an amplitude of the diastolic heart pulse and on an amplitude of the systolic heart pulse.

17. The computer-implemented method of claim 11, further comprising identifying a systolic heart pulse and a diastolic heart pulse from the waveform, wherein identifying a health condition of the user based on the waveform comprises determining a blood pressure of the user based on a time delay of the diastolic heart pulse relative to the systolic heart pulse.

18. The computer-implemented method of claim 11, further comprising filtering, with the waveform, one of a noise component and an interference from an audio signal to a speaker mounted in the in-ear device, and providing, with the speaker, the audio signal to the user.

19. The computer-implemented method of claim 11, further comprising: receiving, from an electrode, an electronic signal indicative of a cardiovascular activity of the user, identifying a correlation of the electronic signal with the waveform, and determining one of a heart rate, a breathing rate, or a blood pressure of the user based on the correlation or a deep neural network algorithm.

20. The computer-implemented method of claim 11, wherein identifying a health condition of the user further comprises performing a spectral analysis on the waveform to identify a p-wave, a QRS-complex, and a T-wave complex in an electro-cardiogram.
